(19) United States
(12) Patent Application Publication
(10) Pub. No.: US 2025/0041518 A1
(43) Pub. Date: Feb. 6, 2025

(54) Insertable Device for Fluid Transmission Having Temperature Sensor

(71) Applicant: Westface Medical Inc., Redwood City, CA (US)

(72) Inventors: Brooks H. Rohlen, Redwood City, CA (US); Sergio Salinas, Redwood City, CA (US); David B. Smith, Redwood City, CA (US)

(21) Appl. No.: 18/665,070

(22) PCT Filed: Nov. 23, 2022
(86) PCT No.: PCT/US2022/050902

§ 371 Date: May 15, 2024

Related U.S. Application Data
(60) Provisional application No. 63/264,601, filed on Nov. 26, 2021.

(51) Int. Cl. A61M 5/172; G16H 20/17; G16H 40/63

(52) U.S. Cl. CPC A61M 5/1723; G16H 20/17; G16H 40/63; A61M 2005/1726; A61M 2230/50

(57) ABSTRACT

Systems and methods for measuring a temperature at a location within a patient's body such as at an in vivo location proximate to a site of intravenous fluid administration, are disclosed. In some embodiments, an insertable device includes an insertion tube member having a tip configured to be inserted into a blood vessel (or other body lumen) of a patient, and a temperature sensor operatively coupled to the insertion tube proximate the tip and configured to measure a temperature within the blood vessel during administration of a fluid through the insertion tube. The device may also include a controller operable to receive one or more signals from the temperature sensor indicative of the temperature within the blood vessel proximate the tip of the insertion tube.

### FIELD OF THE DISCLOSURE

The present disclosure relates generally to devices that are insertable into a patient for fluid transmission for health care diagnosis or treatment, and more specifically, to insertable devices for fluid transmission having a temperature sensor.

### CROSS REFERENCE TO RELATED APPLICATIONS

This patent application claims priority benefits under 35 USC § 119(e) from the following provisional patent application: U.S. Patent Application No. 63/264,601 filed on Nov. 26, 2021, which application is incorporated herein by reference.

### BACKGROUND

For many health care procedures, one or more fluids may be administered to or extracted from a patient's body. To perform such procedures, various insertable devices may be used, such as catheters, syringes equipped with needles, tubes, and other similar devices. Although desirable results have been achieved using prior art devices that are insertable into a patient for fluid transmission, there is considerable room for improvement.

### SUMMARY

Some of the various embodiments of the present disclosure relate to devices that are insertable into a patient for fluid transmission for health care diagnosis or treatment, and more specifically, to insertable devices for fluid transmission having a temperature sensor. Embodiments of insertable devices for fluid transmission that include a temperature sensor in accordance with the present disclosure may advantageously enable a medical practitioner to monitor an in vivo temperature (e.g. core body temperature or local temperature) of the patient during fluid transmission through the insertable device during (and following) a medical procedure. Accordingly, embodiments of insertable devices in accordance with the present disclosure may thereby improve the performance of medical procedures, and may also improve the satisfaction of patients and medical practitioners alike.

For example, in some embodiments, an insertable device includes an insertion tube having a tip configured to be inserted into a lumen of a patient and configured for transmission of a fluid through the insertion tube at least one of into or out of the lumen; and a temperature sensor operatively coupled to the insertion tube proximate the tip and configured to measure a local temperature within the lumen as the fluid flows through the insertion tube at least one of into or out of the lumen. In some embodiments, the temperature sensor includes at least one of a thermistor or a thermocouple.

In further embodiments, the insertable device includes a controller operable to receive one or more signals from the temperature sensor, the controller being operable to process the one or more signals from the temperature sensor to calculate the local temperature within the lumen proximate to the tip of the insertion tube. In some embodiments, the controller is integrated into the insertable device. In some embodiments, the controller is integrated into the temperature sensor of the insertable device. In further embodiments, the controller is remote or distal from the insertable device and wirelessly communicates with the temperature sensor.

In addition, in some embodiments, a system for determining a local temperature during transmission of a fluid comprises an insertable device and a controller. The insertable device includes an insertion tube operatively coupled to a handle, the insertion tube having a tip configured to be inserted into a blood vessel of a patient, and configured for transmission of a fluid through the insertion tube at least one of into or out of the blood vessel, and a temperature sensor operatively coupled to the insertion tube proximate the tip and configured to measure one or more signals indicative of a local temperature within the blood vessel of the patient as the fluid flows through the insertion tube at least one of into or out of the blood vessel. The controller is operatively coupled to the temperature sensor and configured to provide electrical power to the temperature sensor, the controller being configured to process one or more signals received from the temperature sensor to determine the local temperature within the blood vessel proximate to the temperature sensor.

In some embodiments, the controller is configured to process one or more signals received from the temperature sensor to determine the local temperature by one or more operations that correct for a heat transfer effect based on at least one of a fluid flow rate, a fluid temperature, or an initial body temperature of the patient prior to fluid flow. And in further embodiments, the controller is configured to process one or more signals received from the temperature sensor to determine the local temperature by one or more operations that correct for a heat transfer effect by computing a weighted average of a body temperature before fluid flow and a fluid temperature of the fluid passing through the insertion tube.

There has thus been outlined, rather broadly, some of the embodiments of the present disclosure in order that the detailed description thereof may be better understood, and in order that the present contribution to the art may be better appreciated. There are additional embodiments that will be described hereinafter and that will form the subject matter of the claims appended hereto. In this respect, before explaining at least one embodiment in detail, it is to be understood that the various embodiments are not limited in its application to the details of construction or to the arrangements of the components set forth in the following description or illustrated in the drawings. Also, it is to be understood that the phraseology and terminology employed herein are for the purpose of the description and should not be regarded as limiting.

To better understand the nature and advantages of the present disclosure, reference should be made to the following description and the accompanying figures. It is to be understood, however, that each of the figures is provided for the purpose of illustration only and is not intended as a definition of the limits of the scope of the present disclosure. Also, as a general rule, and unless it is evidence to the contrary from the description, where elements in different figures use identical reference numbers, the elements are generally either identical or at least similar in function or purpose.

### BRIEF DESCRIPTION OF THE DRAWINGS

Embodiments of methods and systems in accordance with the teachings of the present disclosure are described in detail below with reference to the following drawings.

FIG. 1 shows a perspective view of an insertable device in accordance with an exemplary embodiment of the present disclosure.

FIG. 2 shows a side elevational view of the insertable device of FIG. 1 in accordance with an exemplary embodiment of the present disclosure.

FIG. 3 shows an enlarged perspective view of a tip of the insertable device of FIG. 1 in accordance with an exemplary embodiment of the present disclosure.

FIG. 4 shows a side elevational view of a handle and an insertion tube of the insertable device of FIG. 1 in accordance with an exemplary embodiment of the present disclosure.

FIG. 5 shows a side elevational view of a portion of an insertable device in accordance with another exemplary embodiment of the present disclosure.

FIG. 6 shows an enlarged, side elevational view of an insertion tube and a head portion of an insertion device in accordance with another exemplary embodiment of the present disclosure.

FIG. 7 shows an embodiment of a fluid administration system that includes an insertable device in accordance with an exemplary embodiment of the present disclosure.

FIG. 8 shows a perspective view of a controller that may be used in accordance with another exemplary embodiment of the present invention.

FIG. 9 shows a perspective view of another controller that may be used in accordance with another exemplary embodiment of the present invention.

FIG. 10 shows another embodiment of a fluid administration system that includes an insertable device in accordance with another exemplary embodiment of the present disclosure.

FIG. 11 shows an embodiment of a method for temperature measurement in accordance with an exemplary embodiment of the present disclosure.

FIG. 12 shows perspective view of a portion of an insertable device in accordance with another exemplary embodiment of the present disclosure.

FIG. 13 shows a schematic view of a wireless interface module in accordance with another exemplary embodiment of the present disclosure.

### DETAILED DESCRIPTION

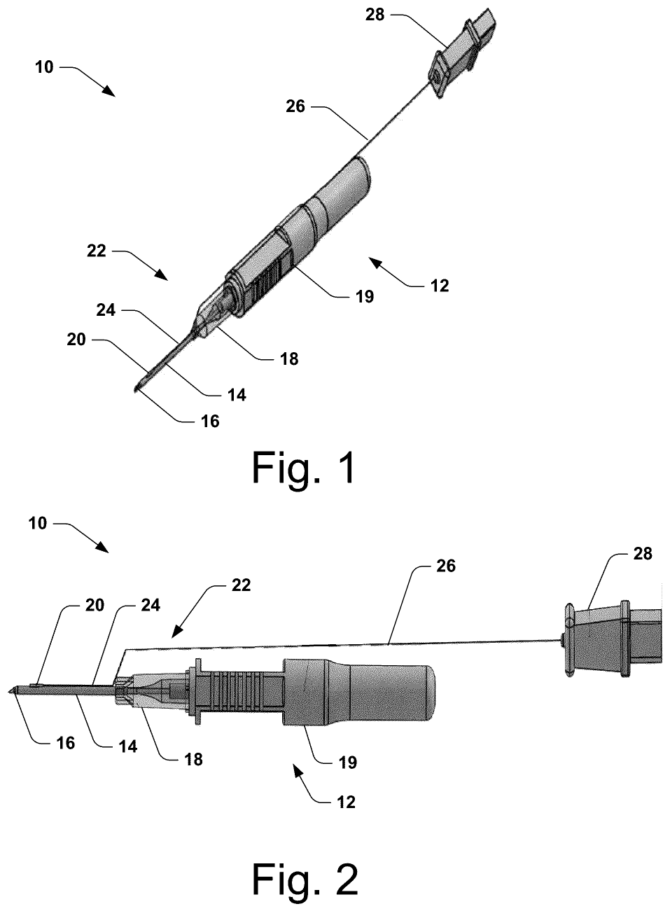

Systems and methods for insertable devices for fluid transmission having a temperature sensor are described herein. Many specific details of certain embodiments are set forth in the following description and in FIGS. 1-13 to provide a thorough understanding of such embodiments. One skilled in the art will understand, however, that the invention may have additional embodiments, or that alternate embodiments may be practiced without several of the details described in the following description.

During some medical procedures, it may be desirable to measure a local temperature at a location within a patient's body proximate to an insertable device, such as, for example, at an in vivo location proximate to a site of intravenous fluid administration. Accordingly, techniques and technologies in accordance with the present disclosure may advantageously provide embodiments of devices that are insertable into a patient for fluid transmission for health care diagnosis or treatment, wherein such insertable devices include a temperature sensor.

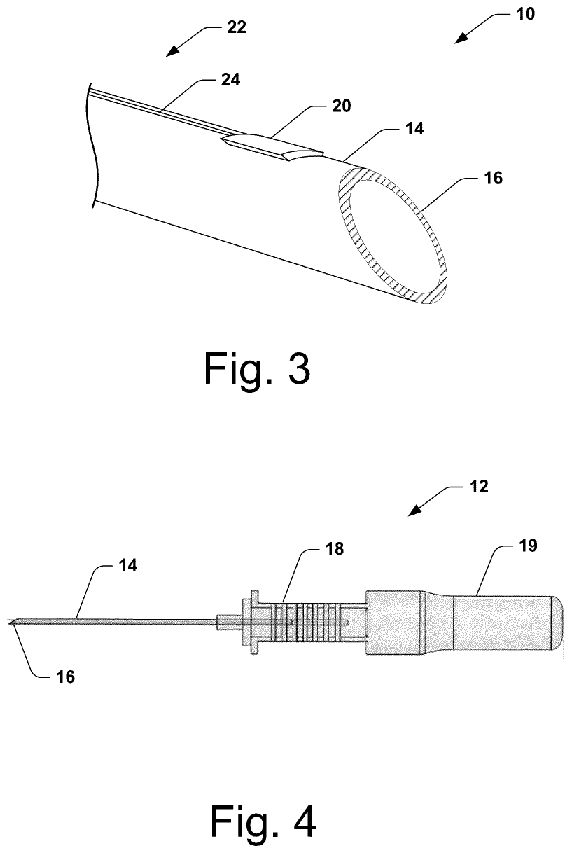

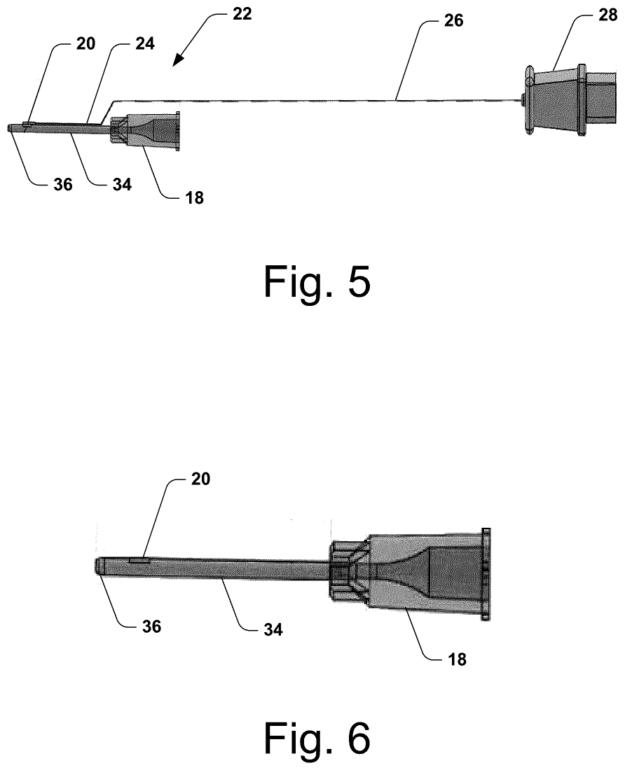

More specifically, FIG. 1 shows a perspective view of an insertable device 10 in accordance with an exemplary embodiment of the present disclosure. FIG. 2 shows a side elevational view of the insertable device 10 of FIG. 1. In this embodiment, the insertable device 10 is configured as a catheter device for transmission of one or more fluids into a patient. More specifically, the insertable device 10 includes a handle 12 operatively coupled to an insertion tube 14 and configured for fluid transmission. In some embodiments, the insertion tube 14 may have a tip 16 that is configured to be inserted into a patient's vein or other vascular lumen. In some embodiments, the insertion tube 14 may be configured as a conventional needle, having a tip 16 that is angled (e.g. as shown in FIGS. 3-4) to provide a sharp point for penetration of a patient's skin. In other embodiments, however, the tip 16 may be configured with a tapered end (or relatively blunt end), without an angle or other sharp points, such as to enable insertion into bodily lumens or orifices while reducing the risk of substantial irritation to surrounding tissues (e.g. as shown in FIGS. 5-6).

As further shown in FIGS. 1-2, the insertable device 10 further includes a temperature sensor 20 attached to the insertion tube 14 proximate the tip 16. In some embodiments, a conductive lead 22 is coupled to the temperature sensor 20, wherein the conductive lead 22 includes a first portion 24 that extends along the insertion tube 14, and a second portion 26 that extends away from the insertion tube 14 to a first connector 28. It will be appreciated that in some embodiments, the temperature sensor 20 and the first portion 24 of the conductive lead 22 may be mounted on an outer surface of the insertion tube 14, while in other embodiments, one or both of the temperature sensor 20 and the first portion 24 of the conductive lead 22 may be embedded into the outer surface of the insertion tube 14.

The temperature sensor 20 may be advantageously configured to sense a local temperature within a patient's body proximate to the tip 16 of the insertion tube 14 while the tip 16 is inserted into the patient's body during a medical procedure. In at least some embodiments, the temperature sensor 20 may be configured such that it is electrically insulated from, yet in thermal equilibrium with, the insertion tube 14 of the insertable device 10.

As further show in FIGS. 1-2, in some embodiments, the handle 12 of the insertable device 10 may include a head portion 18 coupled to the insertion tube 14, and a body portion 19 coupled to the head portion 18. The head portion 18 fluidically couples the body portion 19 with the elongated tube 14 such that fluid entering the body portion 19 can pass through the head portion 18 and out through the insertion tube 14. Alternately, fluid may enter the insertion tube 14, pass through the head portion 18 to the body portion 19, and exit from the body portion 19.

FIG. 4 shows a side elevational view of the handle 12 and insertion tube 14 of the insertable device 10 of FIG. 1 in accordance with an exemplary embodiment. As shown in FIG. 4, in some embodiments, the handle 12 and insertion tube 14 of the insertable device 10 may include a conventional, off-the-shelf catheter assembly. For example, in some particular embodiments, the handle 12 and insertion tube 14 of the insertable device 10 may include a model 18G Safety IV catheter assembly commercially-available from Smiths Medical, a subsidiary of ICU Medical, Inc. of San Clemente, California.

In some embodiments, the head portion 18 and the body portion 19 may be removably coupled. For example, FIG. 5 shows the head portion 18 of the insertable device 10 with the body portion 19 removed. More specifically, in some embodiments, the head portion 18 and the body portion 19 may be removably coupled via a conventional Luer-lock or Luer-slip fitting. In other embodiments, however, the head and body portions 18, 19 may be coupled in a variety of suitable ways, including slip connections, friction connections, screw connections, snap or press-fit connections, or any other suitable coupling methods. In still further embodiments, the head and body portions 18, 19 may be non-removably coupled, and may be integrally-formed as a single, unitary structure.

It will be appreciated that a variety of suitable configurations of insertion tubes may be used. For example, FIG. 6 shows an enlarged, side elevational view of an insertion tube 34 and the head portion 18 in accordance with another exemplary embodiment of the present disclosure. In the embodiment shown in FIGS. 5-6, the insertion tube 34 includes a tip 36 that is not angled as described above (e.g. FIGS. 3-4). In this embodiment, the tip 36 of the insertion tube 34 is tapered such as to enable insertion into bodily lumens or orifices while reducing the risk of substantial irritation to surrounding tissues. Generally, other suitable configurations of insertion tubes may be employed depending upon the preferences of the user or the particular procedure being performed.

It will also be appreciated that a variety of suitable temperature sensors may be used in insertable devices in accordance with the present disclosure. For example, in some embodiments, the temperature sensor 20 may include a thermistor or other similar device that detects temperature based on electrical resistance or variations thereof. In other embodiments, the temperature sensor 20 may include a thermocouple or other similar device that detects temperature based on electrical potential (or capacitance) or variations thereof. In still other embodiments, the temperature sensor 20 may include any other electrical circuit or arrangement of components capable of measuring temperature in any suitable manner.

In some embodiments, the conductive lead 22 allows the application of a voltage across the temperature sensor 20, or alternatively the measurement of a voltage across the temperature sensor 20. For example, if the temperature sensor 20 is comprised of a thermistor, applying a voltage and measuring the resistance across the thermistor enables a user to determine the temperature of the thermistor. Alternatively, if the temperature sensor 20 is comprised of a junction of two different metals as part of a thermocouple, measuring the voltage across the temperature sensor 20 may yield the temperature at the junction.

More specifically, in some particular embodiments, the temperature sensor 20 may include a thermistor having a temperature measurement accuracy ±0.2° C. over a range of measured temperatures from 10° C. to 40° C. In further embodiments, the temperature sensor 20 may include a thermistor having a nominal resistance of ten thousand ohms at 25° C. Of course, in other embodiments, the temperature sensor 20 may include a thermistor having a different operating range, different measurement accuracy, and different nominal resistance value.

Similarly, in some particular embodiments, the first portion 24 of the conductive lead 22 may include a thin metallic wire covered with an insulating material. For example, in some embodiments, the first portion 24 may include a thin wire that includes copper, nickel, nickel alloy, or any other suitable conductive materials. In further particular embodiments, the first portion 24 of the conductive lead 22 may include a #40 AWG, Nickel alloy 200 conductive wire. In some embodiments, the second portion 26 of the conductive lead 22 may include the same materials as the first portion 24. In some other embodiments, the second portion 26 of the conductive lead 22 may be constructed differently from the first portion 24. For example, in some particular embodiments, the second portion 26 of the conductive lead 22 may include a twisted pair of tin-plated copper wires surrounded by an insulative covering, however, in other embodiments, other metals, alloys, or combinations of conductive materials may be used.

As noted above, in some embodiments, the temperature sensor 20 may be positioned on an outer surface of the insertion tube, or alternately, may be embedded into the insertion tube. For example, as further shown in FIG. 6, the temperature sensor 20 is embedded into an outer surface of the insertion tube 34. In some embodiments, the temperature sensor is flush with the outer surface of the insertion tube 34 to further reduce possible irritation to surrounding tissues during insertion of the insertion tube 34 into a patient's body.

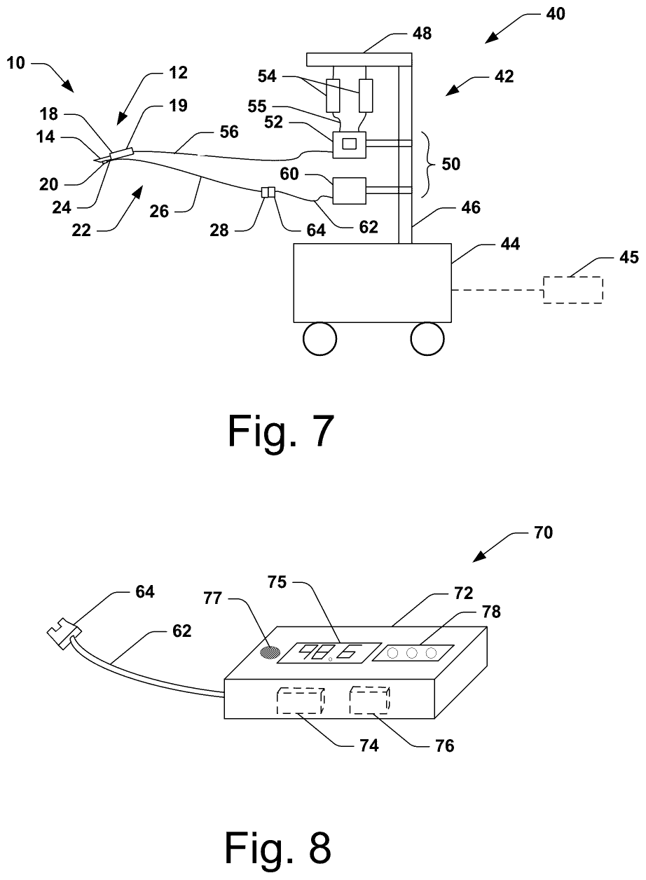

It will be appreciated that techniques and technologies in accordance with the present disclosure may have a variety of suitable uses and applications, including but not limited to the monitoring of temperatures at a location within a patient's body proximate to an insertable device, such as, for example, at an in vivo location proximate to a site of intravenous fluid administration. For example, FIG. 7 shows an embodiment of a fluid administration system 40 that includes the insertable device 10 of FIG. 1 in accordance with an exemplary embodiment of the present disclosure. In some embodiments, the fluid administration system 40 includes an intravenous (IV) tower 42. The IV tower 42 may be a multipurpose device used in medical settings. In some embodiments, the IV tower 42 includes a base 44, a shaft 46 that extends upwardly from the base 44, and a hanger 48 that extends outwardly from the shaft 46.

In some embodiments, the fluid administration system 40 includes a plurality of electronic components 50 that may be affixed to the shaft 46. For example, in some embodiments, the fluid administration system 40 may include an IV pump 52, a controller 60, and one or more other devices as required. In some embodiments, the IV pump 52 may be employed to provide one or more fluids to a patient via the insertable device 10 during a medical procedure. In some embodiments, the IV tower 42 may include an on-board power supply 45, however, in other embodiments, the IV tower 42 may be coupled to an external power supply 45 (e.g. a wall outlet). In some embodiments, the IV tower 42 may provide electrical power to one or more other components of the fluid administration system 40 (e.g. IV pump 52, controller 60, other devices, etc.)

In addition, in some embodiments, the controller 60 may be operatively coupled to the insertable device 10 during the medical procedure, and may be configured to apply one or more electrical signals to the temperature sensor 20 of the insertable device 10, and subsequently to receive one or more signals from the temperature sensor 20 to measure, monitor, and report the temperatures sensed by the temperature sensor 20. More specifically, in some embodiments, a control lead 62 extends from the controller 60 to a second connector 64 that operatively couples with the first connector 28. In some embodiments, the first and second connectors 28, 62 may be of a standardized, conventional design. For example, in some particular embodiments, the first and second connectors 28, 62 may include any of a variety of suitable connectors commercially-available from Molex, LLC of Lisle, Illinois.

With continued reference to FIG. 7, in some embodiments, the fluid administration system 40 further includes one or more IV fluid bags 54 (two shown) that may be affixed to the hanger 48 of the IV tower 42 and coupled to the IV pump 52 by one or more supply lines 55. Similarly, a supply tube 56 extends from the IV pump 52 to the insertable device 10, or more specifically, to the body portion 19 of the handle 12 of the insertable device 10.

In operation, when a fluidic substance is administered to a patient intravenously, the IV bag 54 may be affixed to the hanger 48 of the IV tower 42 and subsequently attached to the IV pump 52. Also, the insertion tube 14 of the insertable device 10 may be inserted into a vein of a patient. The IV pump 52 may be operated to pump the fluid from the IV bag(s) 54 through the supply tube 56 into and through the handle 12, through the insertion tube 14 and into the patient. In alternate embodiments, the IV pump 52 may be eliminated and the fluid may be driven by gravity or other suitable propelling force or mechanism.

In addition, in at least some embodiments, the controller 60 may operate to provide power to the temperature sensor 20, and receive signals from the temperature sensor 20, to measure the local temperature within the patient's body proximate to the tip 16 of the insertable device 10. The controller 60 may also display the temperatures sensed by the temperature sensor 20, or may transmit signals indicating the sensed temperatures to other devices (e.g. the IV pump 52). In some embodiments, the controller 60 and the temperature sensor 20 operate to measure the in vivo temperature simultaneously with the administration of fluid by the fluid administration system 40, however, in alternate embodiments, the temperature measurements may be performed sequentially or consecutively with the administration of fluid.

It will be appreciated that a variety of suitable controllers may be used in association with the insertable device 10. For example, as shown in FIG. 7, the controller 60 may be part of the IV tower 42, however, in some embodiments, the controller may be independent from the IV tower 42 and other components of the fluid administration system 40. For example, FIG. 8 shows an embodiment of a controller 70 that may be used in association with an insertable device (e.g. insertable device 10) in accordance with the present disclosure. In this embodiment, the controller 70 is independent of the IV tower 42, and includes a housing 72 that may encapsulate a power supply 74 and a microcontroller 76. Extending from the housing 72 is the control lead 62 coupled to the second connector 64. The power supply 74 may provide power to the microcontroller 76, the temperature sensor 20, or both. The power supply 74 may also provide power to the other components of the controller 70. In turn, the microcontroller 76 may receive one or more signals (e.g. signals indicative of measured voltage or resistance) from the temperature sensor 20 via the second connector 64 and the control lead 62. In some embodiments, the microcontroller 76 may be configured to convert the received signals from the temperature sensor 20 into temperature measurements.

Referring still to FIG. 8, the controller 70 further includes one or more control buttons 78 that enable a user to interface with the controller 70, including but not limited to turning the controller 70 on and off. In some embodiments, the microcontroller 76 may receive one or more signals from the temperature sensor 20, and may calculate a temperature, and then display the calculated temperature on a visual indicator 75 on the surface of the housing 72. In some embodiments, the microcontroller 76 may also communicate the temperature via an audio indicator 77. For example, in some embodiments, the audio indicator 77 may beep or emit a suitable audible signal when a measurement has been taken, or provide an alert when a temperature measurement is outside a predetermined range.

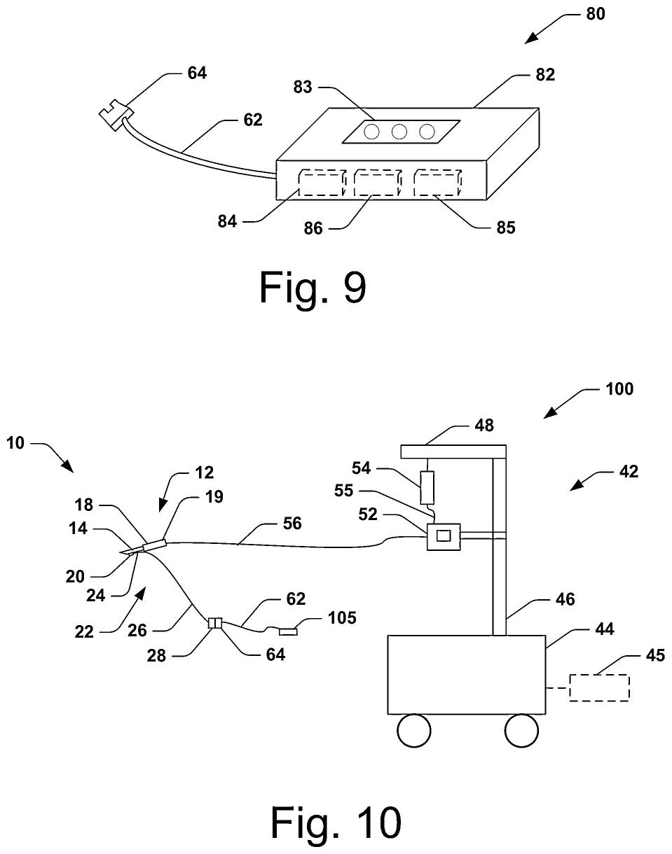

FIG. 9 shows a perspective view of another controller 80 that may be used in accordance with another exemplary embodiment of the present invention. In this embodiment, the controller 80 may receive signals from the temperature sensor 20 wirelessly, and may also transmit the measured temperatures wirelessly to other components. For example, in some embodiments, the controller 80 includes a housing 82 that may encapsulate a power supply 84, a microcontroller 86, and a wireless transmitter 85. Extending from the housing 82 is the control lead 62 coupled to the second connector 64. The controller 80 may further include one or more control buttons 83 that enable a user to interface with the controller 80. As in the previously-described embodiment, the power supply 84 may provide power to the microcontroller 86, the temperature sensor 20, or both. The power supply 84 may also provide power to the wireless transmitter 85 and to the other components of the controller 80.

In some embodiments, the microcontroller 86 may receive one or more signals (e.g. signals indicative of measured voltage or resistance) from the temperature sensor 20 via the second connector 64 and the control lead 62. In alternate embodiments, the wireless transmitter 85 may wirelessly receive one or more signals (e.g. signals indicative of measured voltage or resistance) from the temperature sensor 20, and may provide the received signals to the microcontroller 86. In some embodiments, the microcontroller 86 may be configured to convert the received signals from the temperature sensor 20 into temperature measurements.

In operation, when the microcontroller 86 determines a temperature measurement, it may provide the temperature measurement to the wireless transmitter 85 for wireless transmission from the controller 80 to other components or systems. For example, in some embodiments, the wireless transmitter 85 may communicate via a relatively short-range communication protocol such as Bluetooth^{(R)}. In some embodiments, the wireless transmitter 85 includes a transceiver, however, in further embodiments, the wireless transmitter 85 includes a wireless receiver, a wireless transmitter, or any suitable combination thereof.

FIG. 10 shows another embodiment of a fluid administration system 100 in accordance with another exemplary embodiment of the present disclosure. In this embodiment, the fluid administration system 100 includes several of the same components as the fluid administration system 40 described above (and shown in FIG. 7), however, with some substantial differences. For example, in some embodiments, the fluid administration system 100 includes the intravenous (IV) tower 42 that includes the base 44, the shaft 46 that extends upwardly from the base 44, and the hanger 48 that extends outwardly from the shaft 46. An IV bag 54 hangs from the hanger 48 and provides fluid to the IV pump 52 via a supply line 55. In turn, the IV pump 52 may pump the fluid through the supply line 56 to the handle 12 of the insertable device 10, as described above.

As further shown in FIG. 10, in some embodiments, a controller 105 may be operatively coupled to the insertable device 10 during the medical procedure, and may be configured to apply one or more electrical signals to the temperature sensor 20 of the insertable device 10, and subsequently to receive one or more signals from the temperature sensor 20 to measure, monitor, and report the temperatures sensed by the temperature sensor 20. More specifically, in some embodiments, the control lead 62 extends from the controller 105 to the second connector 64 that operatively couples with the first connector 28.

In at least some embodiments, the controller 105 may include any of the controllers 70, 80 described above and depicted in FIGS. 8-9, and may operate to provide power to the temperature sensor 20, and receive signals from the temperature sensor 20, to measure the local temperature within the patient's body proximate to the tip 16 of the insertable device 10. The controller 105 may also display the temperatures sensed by the temperature sensor 20, or may transmit signals (via wire or wirelessly) indicating the sensed temperatures to other devices (e.g. the IV pump 52, a display device, a monitoring device, etc.). In some embodiments, the controller 105 and the temperature sensor 20 operate to measure the in vivo temperature simultaneously with the administration of fluid by the fluid administration system 100, however, in alternate embodiments, the temperature measurements may be performed sequentially or consecutively with the administration of fluid.

In some embodiments, the controller 105 of the fluid administration system 100 may be affixed to a patient, such as with an adhesive, strap, or any other attachment method. Alternatively, the controller 105 may be placed on a surface next to the patient, such as a bed, table top, shelf, wall, or any other suitable surface. In some embodiments, the controller 105 may be configured for wireless communications with one or more of the temperature sensor 20, the IV pump 52, or any other components (e.g. monitor, display device, etc.). More specifically, in some embodiments, the controller 105 may include a wireless transceiver, a wireless receiver, a wireless transmitter, or any suitable combination thereof.

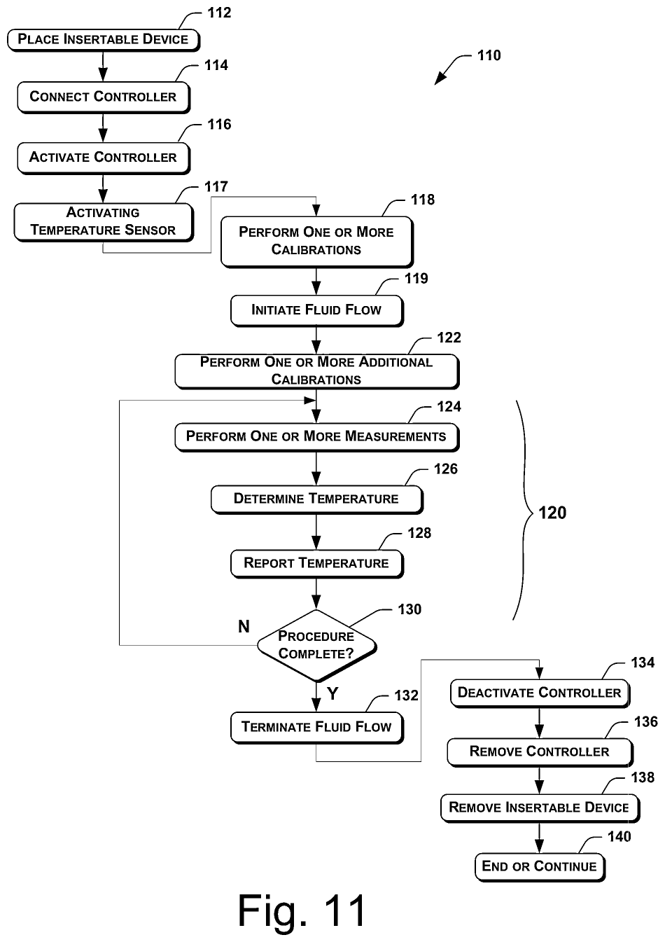

FIG. 11 shows an embodiment of a method for temperature measurement 110 in accordance with an exemplary embodiment of the present disclosure. For the sake of demonstration, the method for temperature measurement 110 may be described in terms of a particular temperature sensor (e.g. wherein the temperature sensor 20 includes a thermistor), or of a particular component of a fluid administration system component (e.g. IV pump 52, controller 60, wireless controller 80, etc.), however, it will be appreciated that any such operations may be suitably modified to accommodate other types of sensors, controllers, or other alternate system components (e.g. thermocouples, or other suitable temperature measurement techniques and technologies, suction devices for withdrawing fluid, etc.). In addition, it will be appreciated that in alternate embodiments, the particular order of some of the operations may be changed, and that some of the operations may be combined, or in some cases omitted, without departing from the scope of the present disclosure. A person having ordinary skill in the art will be able to recognize the modifications to this method necessary in order to develop further embodiments of methods for temperature measure that are still in accordance with the present disclosure.

As shown in FIG. 11, the method 110 includes placing an insertable device into a patient's body at 112. For example, in some embodiments, the insertion tube 14 of the insertable device 10 of FIGS. 1-4 may be inserted by a clinician into a vein of a patient. In other embodiments, an insertion tube (e.g. insertion tube 34) may be inserted into any other suitable bodily lumen or orifice of a patient. In some embodiments, the insertable device 10 may be coupled to the IV pump 52 as shown in FIGS. 7 and 10, or to any other suitable fluid administration systems. Next, the method 110 includes connecting a controller to the insertable device at 114. For example, in some embodiments, the connecting of the controller (at 114) includes coupling the first connector 28 of the insertable device 10 to the second connector 64 that is operatively coupled to the controller 60, thereby closing the circuit between the temperature sensor 20 and the controller 60. In some embodiments, the connecting of the controller (at 114) may further include affixing or placing the controller at a desired location. In some embodiments, such as when the controller 60 is designed to attach to the IV tower 42, the controller 60 will typically be pre-staged on the IV tower 42. Alternatively, if the controller 105 is an integrated controller (e.g. controller 70 of FIG. 8) or wireless controller (e.g. controller 80 of FIG. 9), it may be placed proximate to the patient or affixed with a strap or adhesive to the patient, as desired.

Next, the method 110 further includes activating the controller at 116. In some embodiments, the activating of the controller (at 116) may include operating a switch on the controller to power on the controller (e.g. controller 60) using electrical power provided via the IV tower 42, or alternately, to power on the controller (e.g. controller 70, 80) using electrical power provided by an internal power supply (e.g. power supply 74, 84). In some embodiments, activating the controller (at 116) may further include operatively engaging a wireless communication link between the controller (e.g. controller 80) and any other components of the fluid administration system, such as, for example, the IV pump 52, a display device, the temperature sensor of the insertable device, a monitoring device, or any other suitable devices.

In at least some embodiments, the method 110 further includes activating the temperature sensor of the insertable device at 117. More specifically, in some embodiments, the activating the temperature sensor (at 117) may include applying a voltage to the temperature sensor 20 from the controller 30 via the conductive lead 22 (FIGS. 1-10). In other embodiments, the activating the temperature sensor (at 117) may include any other operations necessary to activate the temperature sensor, such as establishing a wireless communication link with the temperature sensor.

In addition, in at least some embodiments, the method 110 may also include performing one or more calibration operations at 118. For example, in some embodiments, the one or more calibration operations (at 118) may include taking measurements with the temperature sensor of the insertable device prior to initiating fluid flow through the insertable device. More specifically, in some embodiments, one or more calibration measurements may be performed (at 118) in order to establish baseline readings prior to fluid flow through the insertable device, or other similar measurements that may later be used, for example, to perform corrections to improve accuracy of the temperature measurements. Possible calibration operations that may be performed (at 118) are discussed more fully below.

With continued reference to FIG. 11, in at least some embodiments, the method 110 further includes initiating fluid delivery to the patient at 119. For example, in some embodiments, the initiating fluid delivery (at 119) includes operating the IV pump 42 to cause one or more fluids from one or more IV bags 54 to pass through the insertable device 10 and into the patient. Alternately, the one or more fluids may be provided to the patient without use of the IV pump 42. In still other embodiments, the initiating fluid flow (at 119) may include flowing fluids out of a patient, such that the fluid is removed from the patient by entering the insertion tube (e.g. insertion tube 34 of FIGS. 5-6) and passing through the handle 12 of the insertable device 10 and out to a suitable collection vessel.

In at least some embodiments, the method 110 may also include performing one or more additional calibration operations at 122. For example, in some embodiments, the one or more additional calibration operations (at 122) may include taking calibration measurements with the temperature sensor of the insertable device after initiating fluid flow through the insertable device. More specifically, in some embodiments, one or more additional calibration measurements may be performed (at 122) in order to perform corrections that may be used, for example, to improve accuracy of the temperature measurements. Possible additional calibration operations that may be performed (at 122) are discussed more fully below.

As shown in FIG. 11, the method 110 further includes one or more temperature measurement operations at 120. It will be appreciated that the details of the temperature measurement operations (at 120) may vary depending upon the particular configuration of the temperature sensor 20 that is employed on the insertable device, and also based on the particular configuration of the fluid administration system that is being employed in association with the insertable device. In addition, in some embodiments, the temperature measurement operations (at 120) may be performed simultaneously with the flow of fluid through the insertable device, however, in alternate embodiments, the temperature measurement operations (at 120) may be performed sequentially or consecutively with the flow of fluid through the insertable device as desired.

As further shown in FIG. 11, in some embodiments, the temperature measurement operations (at 120) of the method 110 include performing one or more measurements using the temperature sensor at 124. For example, in some embodiments, the performing one or more measurements (at 124) includes measuring a current through a thermistor. In some embodiments, performing one or more measurements (at 124) includes measuring a voltage potential across a thermocouple. It will be appreciated that in some embodiments, the performing one or more measurements (at 124) may be performed by the temperature sensor, wherein the one or more measurements may be processed by the temperature sensor, or may be performed by the temperature sensor and then transmitted to the controller for processing.

In some embodiments, such as when the temperature sensor 20 includes a thermistor, the magnitude of the current across the temperature sensor 20 may depend on the internal temperature of the thermistor. The temperature sensor 20 may initially be at thermal equilibrium with the insertion tube 14, which may in turn be in thermal equilibrium with the patient's blood. In some embodiments, however, since the application of a voltage across the thermistor of the temperature sensor 20 may generate heat, the thermistor temperature may quickly diverge from the blood temperature. To mitigate this effect, in some embodiments, attention may be given during the performing of one or more measurements (at 124) to attempt to minimize this effect. For example, in some embodiments, the performing of one or more measurements (at 124) may include the application of a relatively small voltage to minimize the rate of heat generation by the thermistor. In further embodiments, the performing of one or more measurements (at 124) may include applying voltage for a limited duration to minimize the total heat generated. And in still further embodiments, the performing of one or more measurements (at 124) may include spacing out repeated voltage applications to allow for generated heat to dissipate. One or more of these various possible operational aspects may be performed in order to mitigate a possible negative effect on the temperature measurement due to heating by the temperature sensor.

In some embodiments, the temperature measurement operations (at 120) of the method 110 further includes determining a temperature at 126. For example, in some embodiments, the controller (e.g. controller 60, 70, 80) may receive signals indicating measured values from the temperature sensor of the insertable device (e.g. temperature sensor 20), and may convert or translate the signals into one or more temperature values (at 126). In some embodiments, for example, wherein the temperature sensor includes a thermistor, the determining a temperature (at 126) may include converting a measured current into a temperature value based on a pre-established function. Similarly, in some embodiments, wherein the temperature sensor includes a thermocouple, the determining a temperature (at 126) may include converting a measured voltage potential into a temperature value based on another pre-established function. In some embodiments, the determining a temperature (at 126) may be performed by a controller (e.g. by a microcontroller 76, 86) at a location distal from the temperature sensor. In other embodiments, however, the determining a temperature (at 126) may be performed by the temperature sensor, such as by a microcontroller or other suitable logic circuit that is integrated with the temperature sensor or integrated within the insertable device (e.g. within handle 12). Of course, in other embodiments, the determining a temperature (at 126) may be performed by one or more other suitable components of a fluid administration system.

It will be appreciated that in some embodiments, the determining of the temperature (at 126) may include one or more operations intended to improve an accuracy of the resulting temperature measurement. For example, as noted above, in at least some embodiments, the temperature sensor 20 may be configured such that it is electrically insulated from, yet in thermal equilibrium with, the insertion tube 14 of the insertable device 10. Although such configurations may desirably reduce or eliminate the effects of the temperature of the fluid or the insertion tube 14 on the accuracy of the temperature measurements by the temperature sensor 20, in practice, it may not always be possible to achieve such configurations. Therefore, in some embodiments, it may be desirable to attempt to account for heat transfer phenomena that may impact accuracy of temperature measurements. For example, in some embodiments, it may be desirable to attempt to account for heat transfer phenomena that may be due to the temperature of the fluid passing through the insertion tube 14 in order to improve temperature measurements. Also, in some embodiments, it may be desirable to attempt to account for other effects on temperature measurement accuracy that may be attributable to any other sources.

It will be appreciated that in some embodiments, a device in accordance with the present disclosure may measure a temperature at the site of the temperature sensor and report that temperature as the patient's core temperature without making any further adjustments or corrections. Such embodiments may be practical in various circumstances, such as when a fluid flow is relatively small. In other embodiments, however, a device or system in accordance with the present disclosure may measure a temperature at the site of the temperature sensor, and then make suitable adjustments or corrections to improve the accuracy of the measured temperature (e.g. corrections due to fluid temperature, etc.) in order to provide the desired core body temperature of the patient.

Generally speaking, several relationships between some of the different variables involved in the performance of temperature measurements may be observed. For example, there is typically a correlation between a local temperature measured at the temperature sensor of the insertable device and the patient's core body temperature. In some embodiments, this correlation may assume that the core temperature is higher than the fluid temperature. If the procedure involves pumping fluid that is near or above body temperature, however, some of these relationships will invert. In some embodiments, if the fluid flow rate is zero or relatively small, the local temperature at the temperature sensor and the patient's core temperature are identical or substantially the same for practical purposes. This temperature may therefore be measured (e.g. at 118) prior to initiation of fluid flow (at 119) as a calibration value at the start of the procedure.

In some embodiments, the measured local temperature at the temperature sensor has a positive correlation with both the core temperature and the fluid temperature, and a negative correlation with the fluid flow rate. In other words, in some embodiments, as core temperature increases, local temperature at the temperature sensor increases and vice versa. Similarly, as fluid temperature increases, local temperature at the temperature sensor increases and vice versa. As fluid flow rate increases, however, local temperature at the temperature sensor typically decreases and vice versa (assuming fluid temperature less than core temperature).

Although other variables may be involved, in some embodiments, the local temperature at the temperature sensor may be assumed to be dependent on these three variables (core temperature, fluid temperature, and fluid flow rate). In some embodiments, the local temperature may be described as a weighted average of the core temperature and the fluid temperature. Although the weighting may be defined in various ways as a function of the temperature and flow rate, it will be appreciated that in some embodiments, the weighting may be determined experimentally via one or more calibration measurements at the start of the procedure (e.g. at calibration operations 118, at additional calibration operations 122, or both).

In some embodiments, calibration operations (e.g. at 118) may include taking a measurement of the fluid temperature, or the room temperature as an analog, and assuming this is constant throughout the procedure. In addition, calibration operations (e.g. at 118) may include taking a measurement of the local temperature with no fluid flow, and assuming this to be a direct measurement of the core temperature of the patient. In some embodiments, after fluid flow is initiated (at 119) to a fixed rate, the calibration operations (e.g. at 122) may include waiting several seconds for the local temperature to stabilize, then taking a second measurement of the local temperature. Since the two measurements (at 118 and at 122) may have been taken in close proximity to each other, in some embodiments, it may be assumed that the core body temperature has not changed between the two measurements. It will be appreciated that, in some embodiments, a combination of a local temperature (second calibration measurement at 122), a corresponding core temperature (first calibration measurement at 118), and a fluid temperature may allow calculation of a weighting of the core and fluid temperatures that would yield the measured local temperature (at 124). In some embodiments, the weighting value k is valid only for the current fluid temperature and flow rate. Accordingly, in some embodiments, as the method 110 repeatedly performs temperature measurement operations 120, using the weighting value k and the fluid temperature, measuring the local temperature allows the calculation of the core temperature.

More specifically, in some embodiments, the determining the temperature (at 126) may include one or more operations that assume that the measured temperature is a weighted average of the core body temperature and the temperature of the fluid passing through the insertion tube, as follows:

$$T\_core * k + T\_fluid * ( 1 - k ) = T\_local \qquad ( 1 )$$

where the weighting value k is determined by solving this equation with initial values (e.g. may be determined during one or more calibration operations 118, 122), and where:
T_core=core body temperature measured with no fluid flow,
T_fluid=the fluid temperature (e.g. we may use room temperature as an analog), and
T_local=measured temperature after the fluid is turned on.

In some embodiments, using k and T_fluid, and the measured temperature at T_local, the determining the temperature (at 126) includes solving for T_core (using Equation (1) above), which is the determined core temperature of the patient.

In some embodiments, the weighting value k may be determined (experimentally or theoretically) for a variety of different flow rates and different fluid temperatures, and then stored in the controller (e.g. controller 60, 70, 80) or other suitable portion of the system (e.g. as a lookup table or other suitable database), and that during the fluid flow through the insertable device the actual fluid flow rate and fluid temperature may be measured or determined by one or more components of the fluid administration system (e.g. by IV pump 52, or by a separate flow rate meter or fluid temperature sensor, or manually input by the user, etc.), such as during the one or more additional calibration operations (at 122), or possibly even continuously during the temperature sensing operations (at 120), and based on the actual fluid flow rate and fluid temperature, the weighting value k may be continuously monitored and updated according to the actual values being experienced. More specifically, in some embodiments, one or more monitoring components may be included at one or more appropriate locations (e.g. IV pump 52, insertable device 10, handle 12, supply line 56, etc.) that determines actual fluid flow rate, fluid temperature, or both, of the fluid that is flowing through the insertable device into the patient, and may provide signals to the controller indicative of the determined fluid flow rate and/or fluid temperature for the controller to use in the above-described calculation process.

It will be appreciated that the above-described Equation (1) and methodology is merely one particular embodiment of an attempt to improve the accuracy of the measured temperature using the temperature sensor of the insertable device, and that other corrective calculations may be conceived. In various alternate embodiments, the above-noted corrective calculations may be omitted, or alternative corrective calculations may be employed.

Referring again to FIG. 11, in some embodiments, the temperature measurement operations (at 120) of the method 110 include reporting the temperature at 128. For example, in some embodiments, the reporting the temperature (at 128) may include a controller (e.g. controller 70) visually displaying the calculated temperature on a display (e.g. visual indicator 75). And in some embodiments, the reporting the temperature (at 128) may include a controller (e.g. controller 70) audibly indicating the calculated temperature via an audio indicator (e.g. audio indicator 77). More specifically, in some embodiments, the controller may report the temperature (at 128) visually when the controller is affixed to the IV tower 42 (FIG. 7). In some embodiments, the controller may report the temperature (at 128) by transmitting the temperature, either via a wire or wirelessly, to one or more other components of the fluid administration system (e.g. to IV pump 42, to a display device, to a monitoring device, to a data storage device etc.).

It will be appreciated that the temperature measurement operations (at 120) of the method 110 may be repeated for as long as necessary or desirable. Accordingly, in some embodiments, the method 110 includes determining whether a procedure is complete at 130. If it is determined (at 130) that the procedure is not complete, the method 110 may return to activating the temperature sensor (at 122), and the above-described temperature sensing operations (at 120) may be repeated for as long as desired. Repeat iterations may be activated manually, or scheduled automatically by a controller.

If it is determined (at 130) that the procedure is complete, then in some embodiments, the method 110 may include terminating a fluid flow at 132. The terminating a fluid flow (at 132) may include terminating a flow of fluids either into or out of a patient, such as by powering off the IV pump 42, powering off a vacuum device, or any other suitable procedure. Next, in some embodiments, the method 110 may further include deactivating the controller at 132. In some embodiments, the deactivating of the controller (at 132) may include turning off power to the controller. In further embodiments, the deactivating the controller (at 132) may include disconnecting the controller from the insertable device, such as by disconnecting the second connector 64 from the first connector 28, or any other suitable operation.

In some embodiments, the method 110 may also include removing the controller at 136. For example, in some embodiments, the removing the controller (at 136) may include detaching the controller from the patient's body or other surface to which it has been temporarily attached. In some embodiments, such as when the controller is affixed to the IV tower 42, the removing the controller (at 136) may be omitted, leaving the controller pre-staged on the IV tower 42 for use with the next patient or for the next medical procedure.

As further shown in FIG. 11, in some embodiments, the method 110 further includes removing the insertable device from the patient (at 138). For example, in some embodiments, the removing the insertable device (at 138) includes removing a catheter device from the vein or other portion of the patient's body. Finally, the method 110 may end or continue to other operations at 140.

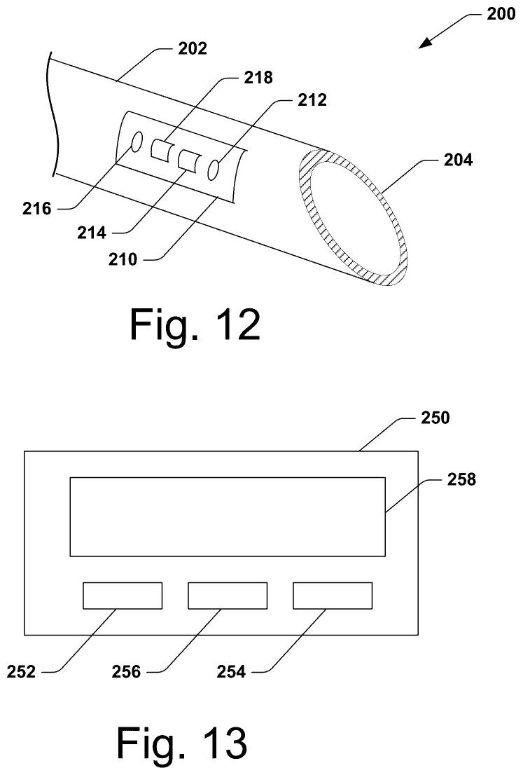

As noted above, in some embodiments, the insertable device may include some on-board processing capability to perform computations, such as to process measured signals (e.g. current, voltage, etc.) into measured temperature values. Such on-board processing may be integrated into, or co-located with, the temperature sensor, or may be located elsewhere within the insertable device. For example, FIG. 12 shows a portion of an insertable device 200 in accordance with another exemplary embodiment of the present disclosure. In this embodiment, the insertable device 200 includes an elongated insertion tube 202 (e.g. a catheter) having a tip 204 (or distal end) that is configured to be inserted into a blood vessel (or other lumen or vessel) of a patient for performing a health care procedure (e.g. administration of a fluid into a blood vessel, extraction of a fluid, etc.). The insertable device 200 may further include a handle and other components as previously described above.

In some embodiments, the insertable device 200 may include an integrated sensor 210 that is mounted on the insertion tube 202 proximate the tip 204, and configured for insertion into the blood vessel or other portion of the patient during performance of a health care procedure. In some embodiments, the integrated sensor 210 may be affixed on a surface of the insertion tube 202, however, in other embodiments, the integrated sensor 210 may be embedded into the insertion tube 202 (e.g. flush mounted). In some embodiments, the integrated sensor 210 includes a temperature sensor 212, a microcontroller 214, a power supply 216, and a wireless transceiver (or transmitter) 218. As described more fully above, the temperature sensor 212 is configured to measure a local temperature within the blood vessel (or other portion of the patient) proximate to the tip 204 of the insertion tube 202. The power supply 216 (e.g. a battery) may be configured to provide power to the other components of the integrated sensor 210 as needed. In some embodiments, the microcontroller 214 may send signals to at least some of the other components of the integrated sensor 210 and may also receive signals from other components of the integrated sensor 210. In some embodiments, the microcontroller 214 may perform on-board processing of signals, such as converting signals from the temperature sensor 212 into temperature measurements. In some embodiments, the transceiver 218 may receive wireless signals from one or more remote sources outside the patient's body, which in turn may be provided to the microcontroller 214 or other components of the integrated sensor 210, and may also transmit wireless signals to one or more remote sites outside the patient's body. In at least some embodiments, the integrated sensor 210 may be configured as a system-on-chip (SOC) configuration, while in other embodiments, the integrated sensor 210 may be any suitable assembly of components sized for insertion into a blood vessel or other in vivo environment within a patient.

In at least some embodiments, the microcontroller 214 may provide one or more signals to the temperature sensor 212 to cause the temperature sensor 212 to perform one or more measurements indicative of the local temperature within the blood vessel. The microcontroller 214 may also receive one or more signals from the temperature sensor 212, and in some embodiments may perform processing of those signals to determine the local temperature within the blood vessel (or other in vivo environment). Similarly, in at least some embodiments, the microcontroller 214 may provide one or more signals to the transceiver 218 which may in turn transmit one or more signals wirelessly from the integrated sensor assembly 210.

FIG. 13 shows a schematic view of a wireless interface module 250 in accordance with another exemplary embodiment of the present disclosure. In this embodiment, the wireless interface module 250 includes a power supply 252 (e.g. a battery or plug into an external power supply), a microcontroller 254, a wireless transceiver (or receiver) 256, and an input/output component 258 that may include various subcomponents such as buttons, an alphanumeric display, switches, keyboard, gauges, meters, speakers antennas, communication ports, etc.

In operation, in at least some embodiments, the microcontroller 254 of the wireless interface module 250 may transmit one or more command signals via the transceiver 256 to the transceiver 218 of the integrated sensor 210, causing the temperature sensor 212 to perform one or more measurements indicative of the local temperature within the patient's body (e.g. blood vessel). In turn, the integrated sensor 210 may transmit one or more signals indicative of the local temperature measured by the integrated sensor 210 via the transceiver 218 to the wireless interface module 250. In at least some embodiments, the wireless interface module 250 may process the received signals as needed (e.g. using the microcontroller 254), and may display the temperature measured by the integrated sensor 210 using the I/O component 258. Alternately, the temperatures may be displayed using the I/O component 258 without any processing by the microcontroller 254 of the wireless interface module 250.

While various embodiments have been described, those skilled in the art will recognize modifications or variations which might be made without departing from the present disclosure. The examples illustrate the various embodiments and are not intended to limit the present disclosure. Therefore, the description and claims should be interpreted liberally with only such limitation as is necessary in view of the pertinent prior art.

Unless otherwise defined, all technical and scientific terms used herein have the same meaning as commonly understood by one of ordinary skill in the art to which this invention belongs. Although methods and materials similar to or equivalent to those described herein can be used in the practice or testing of the various embodiments of the present disclosure, suitable methods and materials are described above. All patent applications, patents, and printed publications cited herein are incorporated herein by reference in their entireties, except for any definitions, subject matter disclaimers or disavowals, and except to the extent that the incorporated material is inconsistent with the express disclosure herein, in which case the language in this disclosure controls. The various embodiments of the present disclosure may be embodied in other specific forms without departing from the spirit or essential attributes thereof, and it is therefore desired that the various embodiments in the present disclosure be considered in all respects as illustrative and not restrictive. Any headings utilized within the description are for convenience only and have no legal or limiting effect.

1. An insertable device, comprising:
a rigid insertion tube having a tip configured to be inserted through a patient's skin and into a lumen of a patient, the rigid insertion tube configured to extend outwardly from the patient's skin to an enlarged head portion that remains external to the patient's skin, the rigid insertion tube and the enlarged head portion being configured for transmission of a fluid through the insertion tube at least one of into or out of the lumen; and
a temperature sensor operatively coupled to the rigid insertion tube proximate the tip and configured to measure a temperature within the lumen as the fluid flows through the insertion tube at least one of into or out of the lumen.

2. The insertable device of claim 1, wherein the rigid insertion tube comprises a needle.

3. The insertable device of claim 1, wherein the enlarged head portion is operatively coupled to a handle, the handle being configured for transmission of the fluid through the handle and the enlarged head and into the rigid insertion tube at least one of into or out of the lumen, the handle being configured to be grasped by a human hand to controllably manipulate the orientation of the rigid insertion tube during insertion of the rigid insertion tube through the patient's skin and into the lumen.

4. The insertable device of claim 1, wherein the handle and the rigid insertion tube are configured as an intravenous catheter, the handle being further configured to be fluidly coupled to a fluid supply for administration of the fluid through the handle and the rigid insertion tube and into the lumen of the patient.

5. The insertable device of claim 1, further comprising a controller operatively coupled to the temperature sensor and configured to receive one or more signals from the temperature sensor, the controller being operable to process the one or more signals from the temperature sensor to calculate the temperature within the lumen proximate to the tip of the rigid insertion tube.

6. The insertable device of claim 1, wherein the tip of the rigid insertion tube comprises an angled tip that includes a sharp point for penetration of the patient's skin.

7. The insertable device of claim 5, wherein the controller is integrated into the temperature sensor of the insertable device and operatively coupled to the rigid insertion tube proximate the tip.

8. The insertable device of claim 5, wherein the controller is distal from the insertable device and wirelessly communicates with the temperature sensor.

9. The insertable device of claim 5, wherein the temperature sensor includes a power source and a transmitter for wirelessly transmitting the one or more signals to the controller.

10. A system for determining a temperature during transmission of a fluid, comprising:
an insertable device having a needle operatively coupled to a handle, the needle including a rigid shaft having a tip configured to be inserted through a patient's skin and into a blood vessel of a patient, the rigid shaft configured to extend outwardly from the patient's skin to an enlarged head portion that remains external to the patient's skin, the rigid shaft and the enlarged head portion being configured for transmission of a fluid through the needle at least one of into or out of the blood vessel;
a temperature sensor operatively coupled to the rigid shaft proximate the tip and configured to measure one or more signals indicative of a temperature within the blood vessel of the patient as the fluid flows through the rigid shaft at least one of into or out of the blood vessel; and
a controller operatively coupled to the temperature sensor and configured to provide electrical power to the temperature sensor, the controller being configured to process one or more signals received from the temperature sensor to determine the temperature within the blood vessel proximate to the temperature sensor.

11. The system of claim 10, further comprising a handle coupled to the enlarged head portion, the handle being configured to be grasped by a human hand to controllably manipulate the orientation of the rigid shaft of the needle during insertion of the rigid shaft through the patient's skin and into the blood vessel.

12. The system of claim 10, wherein the handle and the needle are configured as an intravenous catheter, the handle being further configured to be fluidly coupled to a fluid supply for administration of the fluid through the handle and the needle and into the blood vessel of the patient.

13. The system of claim 10, wherein the temperature sensor includes a power source and a transmitter for wirelessly transmitting the one or more signals to the controller.

14. The system of claim 10, wherein the controller is configured to process one or more signals received from the temperature sensor to determine the temperature by one or more operations that include converting at least one of a measured current or a measured voltage into the local temperature.

15. The system of claim 10, wherein the controller is configured to process one or more signals received from the temperature sensor to determine the temperature by one or more operations that correct for a heat transfer effect based on at least one of a fluid flow rate, a fluid temperature, or an initial body temperature of the patient prior to fluid flow.

16. The system of claim 10, wherein the controller is configured to process one or more signals received from the temperature sensor to determine the temperature by one or more operations that correct for a heat transfer effect by computing a weighted average of a body temperature before fluid flow and at least one of a fluid temperature or a flow rate of the fluid passing through the insertion tube.

17. The system of claim 16, wherein the system further includes a monitoring component that determines at least one of fluid temperature or fluid flow rate, and wherein the controller is configured to receive one or more signals from the monitoring component indicative of at least one of fluid temperature or fluid flow rate, the controller being configured to determine a weighting value based on the at least one of the fluid temperature or the fluid flow rate.

18. The system of claim 10, wherein the controller is configured to perform one or more operations that correct for a heat transfer effect according to
$$T\_core * k + T\_fluid * ( 1 - k ) = T\_local$$
where k is a weighting value determined with initial values,
T_core is a core body temperature measured with no fluid flow,
T_fluid is a fluid temperature, and
T_local is a measured temperature after the fluid is turned on.

19. An insertable device, comprising:
an insertion tube having a tip configured to be inserted through a patient's skin and into a lumen of a patient and configured for transmission of a fluid through the insertion tube at least one of into or out of the lumen;
a temperature sensor operatively coupled to the insertion tube proximate the tip and configured to measure a temperature within the lumen as the fluid flows through the insertion tube at least one of into or out of the lumen; and
a controller operatively coupled to the temperature sensor and configured to provide electrical power to the temperature sensor, the controller being configured to process one or more signals received from the temperature sensor to determine the temperature within the blood vessel proximate to the temperature sensor by one or more operations that correct for a heat transfer effect by computing a weighted average of a body temperature before fluid flow and at least one of a fluid temperature or a flow rate of the fluid passing through the insertion tube.

20. The system of claim 19, wherein the system further includes a monitoring component that determines at least one of fluid temperature or fluid flow rate, and wherein the controller is configured to receive one or more signals from the monitoring component indicative of at least one of fluid temperature or fluid flow rate, the controller being configured to determine a weighting value based on the at least one of the fluid temperature or the fluid flow rate.
